(19) United States
(12) Patent Application Publication
(10) Pub. No.: US 2018/0296816 A1
(43) Pub. Date: Oct. 18, 2018

(54) TRANSDERMAL MICRONEEDLE UNIT AND TRANSDERMAL MICRONEEDLE DRUG DELIVERY DEVICE HAVING THE SAME

(71) Applicant: KIWI Platform Inc. Co., Ltd., New Taipei City (TW)

(72) Inventors: Jung-Tang HUANG, Taipei City (TW); King-Chun CHANG, New Taipei City (TW)

(21) Appl. No.: 16/013,020

(22) Filed: Jun. 20, 2018

Related U.S. Application Data
(63) Continuation-in-part of application No. 15/005,332, filed on Jan. 25, 2016.

(30) Foreign Application Priority Data
Jan. 29, 2015 (TW) 104103104

(51) Int. Cl. A61M 37/00

(52) U.S. Cl. CPC A61M 37/0015; A61M 2037/0023; A61M 2205/3303; A61M 2037/0053; A61M 2205/0216

(57) ABSTRACT

The invention relates to a transdermal microneedle unit and a transdermal microneedle drug delivery device including the transdermal microneedle unit. The transdermal microneedle unit includes a plurality of sheets stacked with each other, each sheet having at least one through hole defined thereon and a barbule arranged at the periphery of the through hole, wherein the through hole on one sheet is penetrated by the barbules of other sheets and the barbules being juxtaposed to form at least one triangular pyramidal transdermal microneedle. The transdermal microneedle drug delivery device comprises a substrate, a transdermal microneedle unit, a union joint and an injection syringe.

### CROSS-REFERENCE TO RELATED APPLICATION

This patent application is a continuation-in-part (CIP) application of U.S. patent application Ser. No. 15/005,332 entitled “Transdermal microneedle unit and transdermal microneedle drug delivery device having the same” filed on Jan. 25, 2016, which claims priority to TW104103104 filed on Jan. 29, 2015. The entire disclosures of the above applications are all incorporated herein by reference.

### BACKGROUND OF THE INVENTION

### Field of the Invention

The present invention relates to a transdermal drug delivery device, especially to a transdermal drug delivery device which may deliver injectable drug to the subcutaneous tissue for treatment.

### Description of the Related Art

The global injectable drug delivery market was valued at $22.5 billion in 2012; it is expected to reach $43.3 billion by 2017 at a CAGR of 14.0% from 2012 to 2017, according to the report of Injectable Drug Delivery Market by Formulations, Devices & Therapeutics—Global Forecasts to 2017, By: marketsandmarkets.com, April 2013, Report Code: BT 1862. The injectable drug delivery technologies market is broadly categorized into two major segments, namely, devices technologies and formulation technologies. Based on product, the injectable drug delivery devices technologies market is further categorized into conventional injection devices, self injection devices, and others (microneedles, nanoneedles and blunt needle injections), while injectable drug delivery formulation technologies market is categorized into conventional drug delivery formulations and novel drug delivery formulations. Conventional injection devices segment accounted for the largest share of the overall injectable drug delivery technologies market in 2012.

In addition, the market is segmented on the basis of its therapeutic applications such as auto immune diseases, hormonal imbalances, oncology, orphan/rare diseases (Hemophilia, Ribose-5-phosphate isomerase deficiency (RPI deficiency), Cystic Fibrosis, and Wilson's disease) and others (pain management, allergies, hepatitis C, and aesthetic treatment). Hormonal disorders commanded the largest share of 50.0% of the global injectable drug delivery market in 2012; it is expected to grow at a CAGR of 13.9% to reach $21.6 billion by 2017. However, auto-immune diseases are the fastest growing segment of this market due to the advent of biologics (tumor necrosis factor (TNF) and Interleukin 1 (IL-1)) and improving patient compliance by the development of self injection devices. As per The American Autoimmune Related Diseases Association, 50 million Americans or 20% of the population or one in five people, are living and managing with auto immune diseases during the year 2013.

The major geographic markets of the injectable drug delivery technologies are North America, Europe, Asia-Pacific, and Rest of the World (RoW). North America dominates the market, followed by Europe. However, Asian and Latin American countries represent the fastest growing markets due to growing number of cancer and diabetes incidences.

In addition, the outbreaks of highly pathogenic avian influenza in Asia for the past few years and spread of the disease worldwide highlight the need to redefine conventional immunization approaches and establish effective mass vaccination strategies to face global pandemics. Vaccination is one of approaches to fight infectious diseases and deaths. The conventional vaccination approach is an invasive method that has disadvantages such as sometimes it is painful for the person, it is required to carry out the injection by medical personnel or professional personnel, the injectable drug delivery is always connected with a risk of infection, and storage and transportation of the vaccine. Transcutaneous immunization (TCI) is a novel route for vaccination, which uses the topical application of vaccine antigens on the skin that can enhance medicine effectiveness and improve patient compliance.

Therefore, the transdermal drug delivery device is worth further developing. Typically, the transdermal drug delivery device has microneedle array that is formed by high precision machining technology, e.g., precision stamping, ion etching, sand blast laser, X-ray laser cutting, lithography, coupled plasma, electrocasting technology. The length of the microneedles typically is about hundreds of micrometers. The transdermal microneedle drug delivery device with minimally invasive piercing can effectively reduce the pain of the users to achieve an injection without pain almost.

In current application, cosmetic surgery using derma roller, also called microneedling therapy system (MTS), is a minimally invasive skin-rejuvenation procedure that involves the use of a device that contains fine needles. The needles are used to puncture the skin to create a controlled skin injury. Each puncture creates a channel that triggers the body to fill these microscopic wounds by producing new collagen and elastin. Through the process of neovascularization and neocollagenesis, there is improvement in skin texture and firmness, as well as reduction in scars, pore size, and stretch marks.

The traditional medical drug delivery technology has its limitations, such as oral dosing is the most convenient and cheapest way, but the medical drug absorption is interfered by diet and other drug. Also, the absorbed dose of the medical drug is reduced due to hepatic metabolism. As to intravenous injection, the drug delivery may be fast and accurate, but it is required to provide by the professional and painful for patients. In medical applications, the transdermal drug delivery device with microneedle array can deliver drugs through the skin, and can penetrate drugs through the skin into the bloodstream, is a very attractive and new drug delivery technology.

The array-arranged microneedles of a transdermal drug delivery device can be manufactured with standard semiconductor process such as photolithograph process and etching process. The related art disclosed a process for manufacturing silicon microneedles. Firstly a silicon wafer with a first patterned photoresist layer is prepared. Next, a through hole is defined on the wafer by anisotropic etching. Afterward, a chromium layer is coated on the wafer and a second patterned photoresist layer is formed atop the through hole to function as circular etching mask. Next, the wafer is then etched to form outer tapered wall for the microneedles. However, the silicon-based microneedles are brittle and tend to break when the microneedles prick through user's skin.

Alternatively, hollow microneedles with resin barbules are proposed, where the barbules are drilled by laser processing. Firstly, sheet with barbules is formed by extruding polyimide or polyether ether ketone, and then the barbules are drilled by laser to form hollow microneedles. However, the microneedles have compact size such that the barbules may have ragged edge after extrusion. Moreover, it is difficult to form a hollow microneedle with off-axis through hole or central through hole having uniform inner diameter by laser processing.

In summary, there is a need to provide a transdermal drug delivery device which may deliver injectable drug to the subcutaneous tissue for treatment. The microneedle of the transdermal drug delivery device can be kept intact after the microneedle pricks user's skin for drug delivery.

### SUMMARY OF THE INVENTION

One object of the present invention is to provide a transdermal microneedle unit, where the transdermal microneedle unit has microneedles made by punching or etching to have sufficient mechanical strength. The microneedle is formed by barbules having different aspects to juxtapose each other after the sheets are stacked together, and the tips of the barbules are in a polygon arrangement from top view. The microneedle can be kept intact after the microneedle pricks user's skin for drug delivery.

Accordingly, the present invention provides a transdermal microneedle unit comprising: a plurality of sheets stacked with each other, each of sheets having a through hole defined thereon and a barbule arranged at the periphery of the through hole, wherein the through hole on one sheet is penetrated by the barbules of other sheets, and all the barbules juxtapose with each other to form a transdermal microneedle, and the tips of the barbules are in a polygon arrangement from top view.

In an aspect of the invention, the transdermal microneedle unit comprises a first sheet and a second sheet stacked with the first sheet. The first sheet has a first through hole defined thereon, and a first barbule at the periphery of the first through hole. The second sheet has a second through hole defined thereon, and a second barbule at the periphery of the second through hole, where the second barbule penetrates the first through hole to juxtapose the first barbule.

In another aspect of the invention, the transdermal microneedle unit comprises a first sheet, a second sheet and a third sheet stacked with each other. The first sheet has a first through hole defined thereon, and a first barbule at the periphery of the first through hole. The second sheet has a second through hole defined thereon, and a second barbule at the periphery of the second through hole. The third sheet has a third through hole defined thereon, and a third barbule at the periphery of the third through hole. The second barbule and the third barbule penetrates the first through hole to juxtapose the first barbule, and the tips of the barbules are in triangular arrangement from top view.

In still another aspect of the invention, the transdermal microneedle unit comprises a first sheet, a second sheet, a third sheet and a fourth sheet stacked with each other. The first sheet has a first through hole defined thereon, and a first barbule at the periphery of the first through hole. The second sheet has a second through hole defined thereon, and a second barbule at the periphery of the second through hole. The third sheet has a third through hole defined thereon, and a third barbule at the periphery of the third through hole. The fourth sheet has a fourth through hole defined thereon, and a fourth barbule at the periphery of the fourth through hole. The second barbule, the third barbule and the fourth barbule penetrates the first through hole to juxtapose the first barbule, and the tips of the barbules are in rectangular arrangement from top view.

The transdermal microneedle unit has a first barbule comprising a tip and a base. The tips of those barbules, after the sheets are stacked together, are not at the same altitudes to form an opening for medications passing through. Namely, some barbules pass more through holes than other barbules. Alternatively, the height of the barbules can be such designed, based on the stacked order of sheets, that the tips of those barbules, after the sheets are stacked together, are at the same altitudes to form an opening by cutting at least one tip of the barbule for medications passing through.

The barbules of the transdermal microneedle are made by punching, etching, molding, micromachining, hot forming or cold forming. The barbule of the transdermal microneedle has a material selected from stainless steel, nickel, nickel alloy, titanium, titanium alloy, carbon nanotube, silicon or resin. In case that the biological incompatible material is used, the surface of the barbule may be coated with a layer of biological compatible material.

In order to achieve the object of the present invention, the present invention provides a transdermal microneedle unit comprising a plurality of sheets stacked with each other, each of sheets having array-arranged through holes defined thereon and a barbule arranged at the periphery of each the through holes in array arrangement, wherein the array-arranged through holes on one sheet is penetrated by the barbules of other sheets, and all the barbules juxtapose with each other to form a transdermal microneedle, and the tips of the barbules are in a polygon arrangement from top view. Every barbule has the same aspect on a sheet, or the barbules in different row have different aspects on a sheet. The transdermal microneedle unit is combined with a substrate, and there is a space surrounded by the barbules of the transdermal microneedle unit for embedding with a low flowability medication.

Another object of the present invention is to provide a transdermal microneedle drug delivery device. The transdermal microneedle drug delivery device may deliver injectable drug to the subcutaneous tissue for treatment.

Accordingly, the present invention provides a transdermal microneedle drug delivery device comprising a substrate, a transdermal microneedle unit and a union joint. The transdermal microneedle unit is provided on the substrate, and the transdermal microneedle unit comprises a plurality of sheets stacked with each other, each of sheets having at least one through hole defined thereon and a barbule arranged at the periphery of the through hole, wherein the through hole on one sheet is penetrated by the barbules of other sheets, and all the barbules juxtapose with each other to form a transdermal microneedle, and the tips of the barbules are in a polygon arrangement from top view. The transdermal microneedles of the transdermal microneedle unit may be arranged in array arrangement. The union joint is connected with the substrate by an end thereof, and connected with an injection syringe by another end to apply the medications into skin. The union joint has a circular groove in the front surface of an end thereof, and an O-ring is provided in the circular groove of the union joint in order to avoid a leakage of medications.

The transdermal microneedle drug delivery device of the invention further comprises a gasket which has at least one projecting part for sealing an opening on the bottom of the transdermal microneedle of the transdermal microneedle unit. The gasket is an insert molding article formed by injection molding. Alternatively, the gasket is molded independently, thereafter the gasket is combined with the transdermal microneedle unit.

In the transdermal microneedle drug delivery device of the invention, the substrate has a plurality of latches, and each of latches has an entrance at an end thereof, and the union joint has a plurality of projections at a side surface of an end, and the union joint is engaged with the substrate by screwing each of projections into the corresponding entrances of the latches.

The transdermal microneedle drug delivery device further comprises an injection syringe including a plunger, in which the injection syringe has a connecting end for connecting with another end of the union joint, and the plunger is pushed along inside a cylindrical tube of the injection syringe to apply the medications into skin. The transdermal microneedle drug delivery device with minimally invasive piercing can effectively reduce the pain of the users to achieve an injection without pain almost.

In addition, the transdermal microneedle drug delivery device further comprises a micropump and a micro control unit, in which the micropump is connected with another end of the union joint, and the micropump is driven by a signal produced from the micro control unit to apply the medications into skin. The transdermal microneedle drug delivery device with minimally invasive piercing can effectively reduce the pain of the users to achieve an injection without pain almost.

Compared to the prior art, the transdermal microneedle unit of the invention has microneedles made by punching or etching to have sufficient mechanical strength. The microneedle can be kept intact after the microneedle pricks user's skin for drug delivery. In addition, the method for manufacturing the transdermal microneedle unit is simple for mass production.

### BRIEF DESCRIPTION OF THE DRAWINGS

FIG. 1 shows an exploded view of the transdermal microneedle unit according to an embodiment of the present invention from a front viewing direction.

FIG. 2 is a top view of the transdermal microneedle of the transdermal microneedle unit according to an embodiment of the present invention.

FIG. 3 is a top view of the transdermal microneedle of the transdermal microneedle unit according to another embodiment of the present invention.

FIG. 4 is a top view of the transdermal microneedle of the transdermal microneedle unit according to still another embodiment of the present invention.

FIG. 5 is a top view of the transdermal microneedle of the transdermal microneedle unit according to further another embodiment of the present invention.

FIG. 6 shows an exploded view of the transdermal microneedle unit according to another embodiment of the present invention from a front viewing direction, wherein every barbule has the same aspect on a sheet.

FIG. 7 shows an assembled view of the transdermal microneedle unit of FIG. 6.

FIG. 8 shows an exploded view of another transdermal microneedle unit according to an embodiment of the present invention from a front viewing direction, wherein barbules in different row have different aspects on a sheet.

FIG. 9 shows an exploded view of the transdermal microneedle unit and a gasket according to an embodiment of the present invention from a front viewing direction, wherein the gasket is an insert molding article formed by injection molding.

FIG. 10 shows an exploded view of the transdermal microneedle unit and a gasket according to an embodiment of the present invention from a front viewing direction, wherein the gasket is an independent molding article for combining with the transdermal microneedle unit.

FIG. 11 shows an assembled view of a first sheet, second sheet and third sheet of the transdermal microneedle unit and a gasket according to an embodiment of the present invention from a front viewing direction.

FIG. 12 shows a top view of an assembled view of a first sheet, second sheet and third sheet of the transdermal microneedle unit and a gasket according to an embodiment of the present invention.

FIG. 13 shows an exploded view of a transdermal microneedle drug delivery device according to an embodiment of the present invention from a front viewing direction.

FIG. 14 shows an exploded view of a transdermal microneedle drug delivery device according to an embodiment of the present invention from a rear viewing direction.

FIG. 15 shows an assembled view of a substrate and a union joint according to an embodiment of the present invention from a rear viewing direction.

FIG. 16 shows a schematic view of a transdermal microneedle drug delivery device and an injection syringe in disassembled state according to an embodiment of the present invention.

FIG. 17 shows a sectional assembled view of a transdermal microneedle drug delivery device and an injection syringe for applying drug according to an embodiment of the present invention.

FIG. 18 shows an exploded view of a transdermal microneedle unit and an adhesive film according to an embodiment of the present invention.

FIG. 19 shows a bottom view of the assembly of FIG. 18.

FIG. 20 shows a cross-sectional view of the assembly of FIG. 18.

FIG. 21 shows an exploded view of a transdermal microneedle drug delivery device according to another embodiment of the present invention.

FIG. 22 shows an exploded view of an injection syringe and a drug delivery device according to another embodiment of the present invention.

FIG. 23 shows an exploded view of a drug delivery device according to a further embodiment of the present invention.

FIG. 24 shows an exploded view of a drug delivery device in accordance to another embodiment of the present invention.

FIG. 25 shows an exploded view of a drug delivery device in accordance to a further embodiment of the present invention.

### DETAILED DESCRIPTION OF THE INVENTION

The features of the invention believed to be novel are set forth with particularity in the appended claims. The invention itself, however, may be best understood by reference to the following detailed description of the invention, which describes an exemplary embodiment of the invention, taken in conjunction with the accompanying drawings, in which:

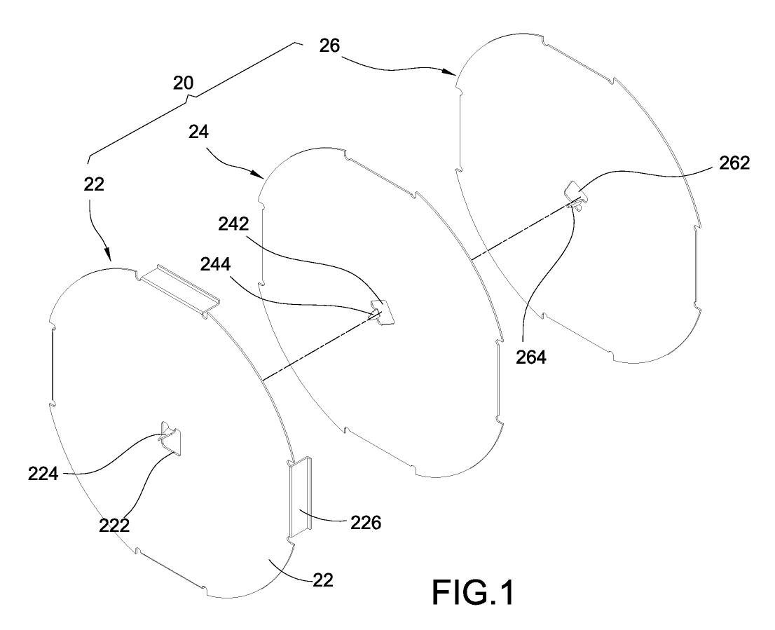

FIG. 1 shows an exploded view of the transdermal microneedle unit according to an embodiment of the present invention from a front viewing direction. According to FIG. 1, the transdermal microneedle unit 20 comprises a first sheet 22, a second sheet 24 and a third sheet 26 stacked with each other. The first sheet 22 has a first through hole 222 defined thereon, and a first barbule 224 at the periphery of the first through hole 222. The second sheet 24 has a second through hole 242 defined thereon, and a second barbule 244 at the periphery of the second through hole 242. The third sheet 26 has a third through hole 262 defined thereon, and a third barbule 264 at the periphery of the third through hole 262. The second barbule 244 and the third barbule 264 penetrates the first through hole 222 to juxtapose the first barbule 224, and the tips of the barbules are in isosceles triangular arrangement from top view. Although the embodiment of FIG. 1 illustrates the transdermal microneedle unit is formed by three sheets each has a through hole and a barbule stacked with each other, in the other embodiments it may be formed by two sheets each has a through hole and a barbule stacked with each other, where the second barbule penetrates the first through hole to juxtapose the first barbule, or it may be formed by four sheets each has a through hole and a barbule stacked with each other, where the second barbule, the third barbule and the fourth barbule penetrate the first through hole to juxtapose the first barbule, and the tips of the barbules are in rectangular arrangement from top view.

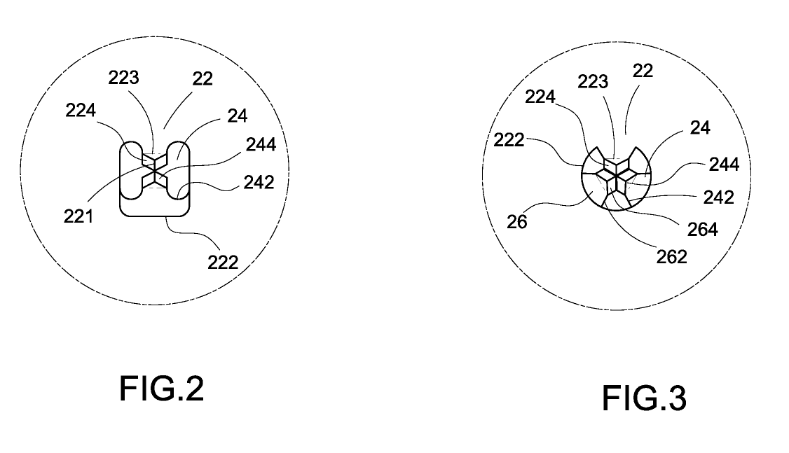

With reference to FIGS. 2 to 5, FIG. 2 is a top view of the transdermal microneedle of the transdermal microneedle unit according to an embodiment of the present invention. According to FIG. 2, the transdermal microneedle unit 20 comprises a first sheet 22 and a second sheet 24 stacked with the first sheet 22. The first sheet 22 has a first through hole 222 defined thereon, and a first barbule 224 at the periphery of the first through hole 222. The second sheet 24 has a second through hole 242 defined thereon, and a second barbule 244 at the periphery of the second through hole 242, where the second barbule 244 penetrates the first through hole 222 to juxtapose the first barbule 224.

FIG. 3 is a top view of the transdermal microneedle of the transdermal microneedle unit according to another embodiment of the present invention. According to FIG. 3, the transdermal microneedle unit 20 comprises a first sheet 22, a second sheet 24 and a third sheet 26 stacked with each other. The first sheet 22 has a first through hole 222 defined thereon, and a first barbule 224 at the periphery of the first through hole 222. The second sheet 24 has a second through hole 242 defined thereon, and a second barbule 244 at the periphery of the second through hole 242. The third sheet 26 has a third through hole 262 defined thereon, and a third barbule 264 at the periphery of the third through hole 262. The second barbule 244 and the third barbule 264 penetrates the first through hole 222 to juxtapose the first barbule 224, and the tips of the barbules are in right triangular arrangement from top view.

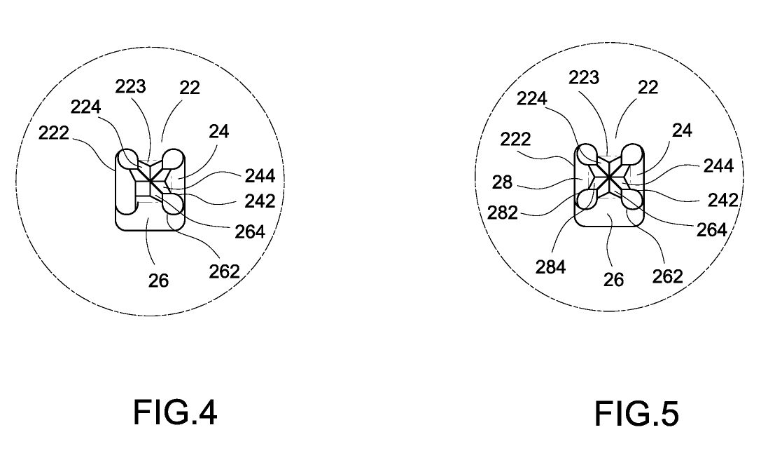

FIG. 4 is a top view of the transdermal microneedle of the transdermal microneedle unit according to still another embodiment of the present invention. According to FIG. 4, the transdermal microneedle unit 20 comprises a first sheet 22, a second sheet 24 and a third sheet 26 stacked with each other. The first sheet 22 has a first through hole 222 defined thereon, and a first barbule 224 at the periphery of the first through hole 222. The second sheet 24 has a second through hole 242 defined thereon, and a second barbule 244 at the periphery of the second through hole 242. The third sheet 26 has a third through hole 262 defined thereon, and a third barbule 264 at the periphery of the third through hole 262. The second barbule 244 and the third barbule 264 penetrates the first through hole 222 to juxtapose the first barbule 224, and the tips of the barbules are in isosceles triangular arrangement from top view.

FIG. 5 is a top view of the transdermal microneedle of the transdermal microneedle unit according to further another embodiment of the present invention. According to FIG. 5, the transdermal microneedle unit 20 comprises a first sheet 22, a second sheet 24, a third sheet 26 and a fourth sheet 28 stacked with each other. The first sheet 22 has a first through hole 222 defined thereon, and a first barbule 224 at the periphery of the first through hole 222. The second sheet 24 has a second through hole 242 defined thereon, and a second barbule 244 at the periphery of the second through hole 242. The third sheet 26 has a third through hole 262 defined thereon, and a third barbule 264 at the periphery of the third through hole 262. The fourth sheet 28 has a fourth through hole 282 defined thereon, and a fourth barbule 284 at the periphery of the fourth through hole 282. The second barbule 244, the third barbule 264 and the fourth barbule 284 penetrates the first through hole 222 to juxtapose the first barbule 224, and the tips of the barbules are in rectangular arrangement from top view.

With the four embodiments as shown in FIGS. 2 to 5, the transdermal microneedle unit 20 has a first barbule 224 comprising a tip 221 and a base 223. The tips of those barbules, after the sheets are stacked together, are not at the same altitudes to form an opening. Namely, some barbules pass more through holes than other barbules. Alternatively, the height of the barbules can be such designed, based on the stacked order of sheets, that the tips of those barbules, after the sheets are stacked together, are at the same altitudes to form an opening by cutting at least one tip of the barbule.

Please refer to FIG. 1 again. In an embodiment, the barbules 224, 244, 264 of the transdermal microneedle unit 20 can be made by punching, etching, molding, micromachining, hot forming or cold forming process. The material of the barbules 224, 244, 264 is selected from the group consisting of stainless steel, nickel, nickel alloy, titanium, titanium alloy, carbon nanotube, and silicon. Alternatively, the material of the barbules can also be selected from the group consisting of polycarbonate, polymethacrylic acid copolymer, ethylene vinyl acetate copolymer, polytetrafluoroethylene, and polyester. Also, the barbules 224, 244, 264 of the transdermal microneedle unit 20 can be made by injection molding or hot pressing. Moreover, the height of the barbules 224, 244, 264 is 300-2500 micrometers; the base width of the barbules 224, 244, 264 is 150-650 micrometers. The separation between tips of the barbules 224, 244, 264 is 500-2000 micrometers. The opening of the microneedles is off-axis with an equivalent diameter of 20-100 micrometers.

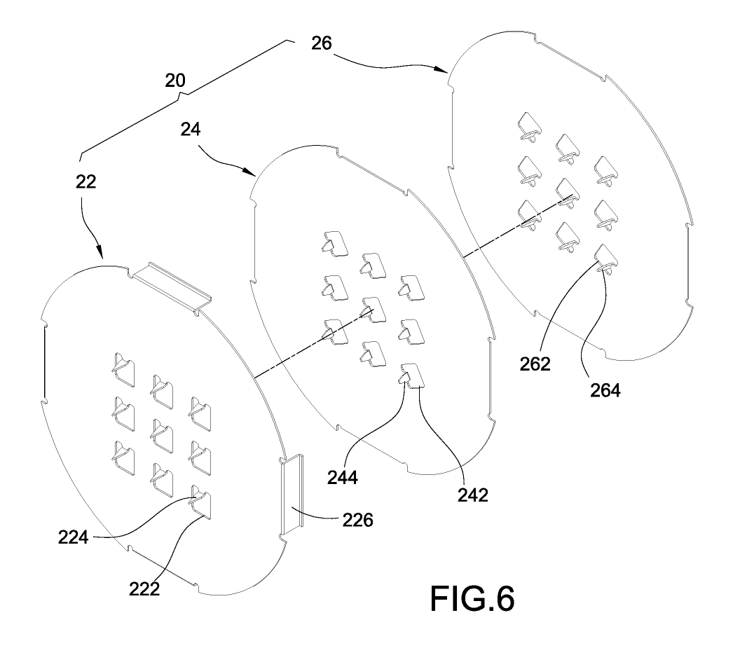

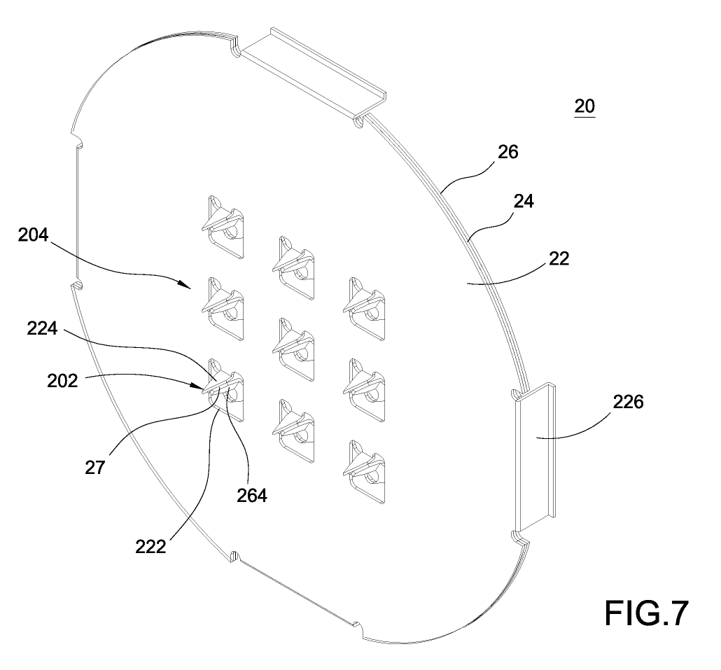

Next, please refer to FIG. 6 and FIG. 7. FIG. 6 shows an exploded view of the transdermal microneedle unit according to another embodiment of the present invention from a front viewing direction, wherein every barbule has the same aspect on a sheet. FIG. 7 shows an assembled view of the transdermal microneedle unit of FIG. 6. In an embodiment, the transdermal microneedle unit 20 comprises a first sheet 22, a second sheet 24 and a third sheet 26 stacked with each other. Each of the first sheet 22, the second sheet 24 and the third sheet 26 has array-arranged through holes 222, 242, 262 defined thereon, and a first barbule 224 at the periphery of the first through hole 222, a second barbule 244 at the periphery of the second through hole 242 and a third barbule 264 at the periphery of the third through hole 262. The second array-arranged barbules 244 of the second sheet 24 and the third array-arranged barbules 264 of the third sheet 26 penetrate the first array-arranged through holes 222 of the first sheet 22 in correspondent position to juxtapose the first array-arranged barbules 224 for forming an array-arranged transdermal microneedle 204 as shown in FIG. 7.

Please refer to FIG. 7 again. In another embodiment, the transdermal microneedle unit 20 of the present invention can combine with the latter-mentioned substrate, and the transdermal microneedle 202 of the transdermal microneedle unit 20 is formed of a first barbule 224, a second barbule 244 and a third barbule 264, and there is a space 27 surrounded by the first barbule 224, the second barbule 244 and the third barbule 264. The space 27 can be embedded with a low flowability medication. The barbules are hard, and particularly may made by stainless steel having a thickness less than 0.05 mm to increase the space that can contain medication. Because the surfaces of the barbules are not required to be electroplated with gold or silver, the cost can be greatly reduced, and the transdermal microneedle unit 20 can have more feeding area, for example area of 1 cm×1 cm with 3×3, 4×4, 5×5, 6×6, 7×7 and 8×8 array-arranged micro-needles, or 4×4 array-arranged micro-needles having only 12 micro-needles at four edges or micro-needles arranged circularly, or area of 2 cm×2 cm with 16×16 array-arranged micro-needles. The dose of the array-arranged micro-needles can be increased. The array-arranged micro-needles can be used to deliver pain-killer, e.g., morphine since it is discarded to avoid infection. Also, the array-arranged micro-needles can be used to deliver chronic medications, e.g., alternatives of smoking addition.

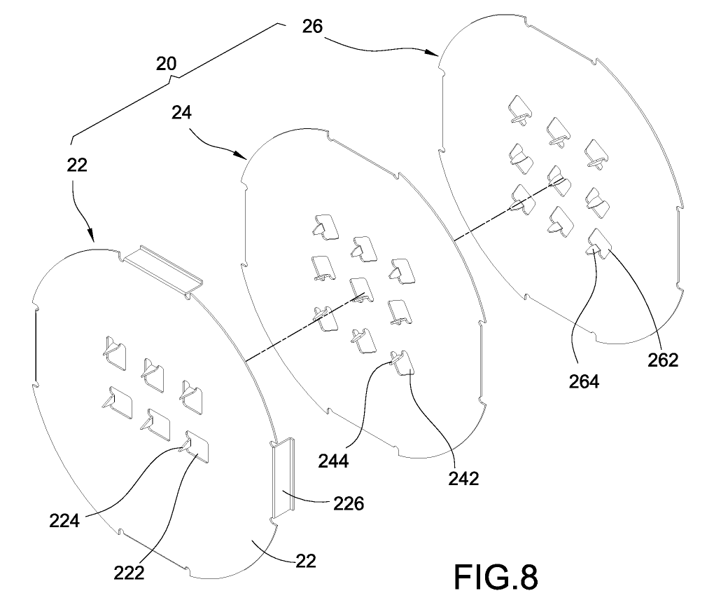

FIG. 8 shows an exploded view of another transdermal microneedle unit according to an embodiment of the present invention from a front viewing direction, wherein barbules in different row have different aspects on a sheet. The difference between FIG. 8 and FIG. 6 is that an embodiment of FIG. 8 has the barbules in different row have different aspects on a sheet.

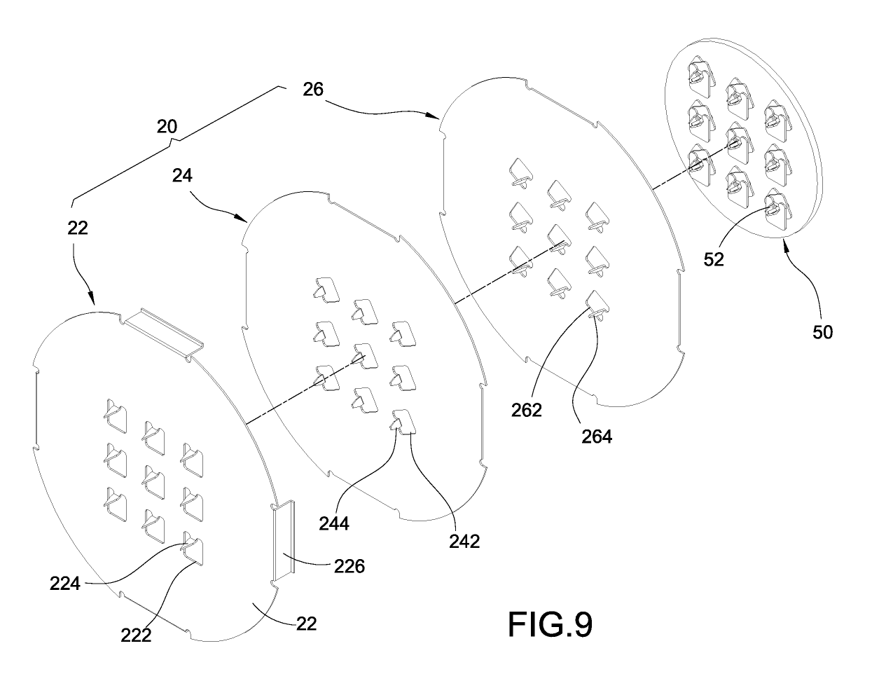

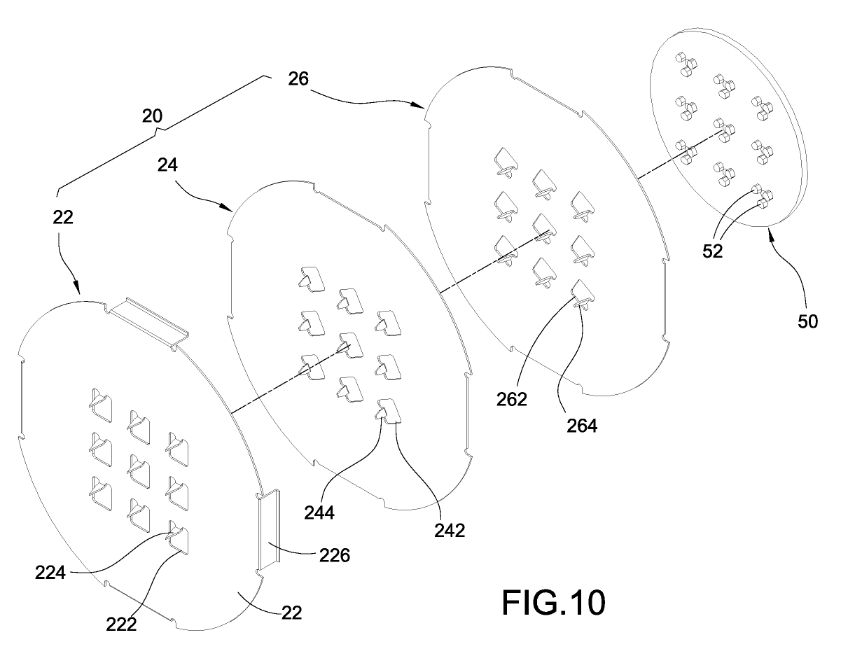

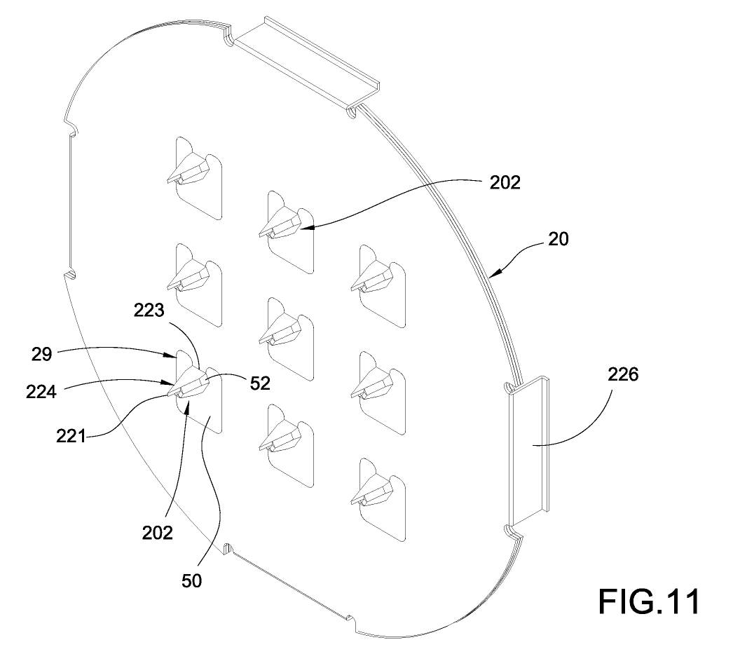

Please refer to FIGS. 9, 10 and 11. FIG. 9 shows an exploded view of the transdermal microneedle unit and a gasket according to an embodiment of the present invention from a front viewing direction, wherein the gasket is an insert molding article formed by injection molding. FIG. 10 shows an exploded view of the transdermal microneedle unit and a gasket according to an embodiment of the present invention from a front viewing direction, wherein the gasket is an independent molding article for combining with the transdermal microneedle unit. FIG. 11 shows an assembled view of a first sheet, second sheet and third sheet of the transdermal microneedle unit and a gasket according to an embodiment of the present invention from a front viewing direction. FIGS. 9 and 10 have the transdermal microneedle unit 20 the same with that of FIG. 6. In an embodiment of FIGS. 9 and 10, the transdermal microneedle unit 20 comprises a first sheet 22, a second sheet 24 and a third sheet 26 stacked with each other. Each of the first sheet 22, the second sheet 24 and the third sheet 26 has array-arranged through holes 222, 242, 262 defined thereon, and a first barbule 224 at the periphery of the first through hole 222, a second barbule 244 at the periphery of the second through hole 242 and a third barbule 264 at the periphery of the third through hole 262. The second array-arranged barbules 244 of the second sheet 24 and the third array-arranged barbules 264 of the third sheet 26 penetrate the first array-arranged through holes 222 of the first sheet 22 in correspondent position to juxtapose the first array-arranged barbules 224 for forming an array-arranged transdermal microneedle.

Please refer to FIG. 11 again. The transdermal microneedles 202 of the transdermal microneedle unit 20 can be arranged to form 3×3 array-arranged micro-needles. In addition, the barbs 226 may be provided on the edge of the first sheet 22 to engage with the grooves 105 of the latter-mentioned substrate 10.

Please refer to FIGS. 9 and 11 again. In an embodiment, the gasket 50 has at least one projecting part 52. The transdermal microneedle unit 20 has an opening 29 on the bottom through where the projecting part 52 of the gasket 50 may penetrate to seal the opening 29 effectively in order to avoid a leakage of medications. According to FIG. 9, the gasket 50 is made by injection molding to combine with the transdermal microneedle unit 20 simultaneously. In other words, the gasket 50 is an insert molding article which can combine with the transdermal microneedle unit 20 closely to avoid a leakage of medications from the opening 29. Alternatively, according to FIG. 10, after the gasket 50 is molded independently, the gasket 50 may combine with the transdermal microneedle unit 20 to avoid a leakage of medications from the opening 29.

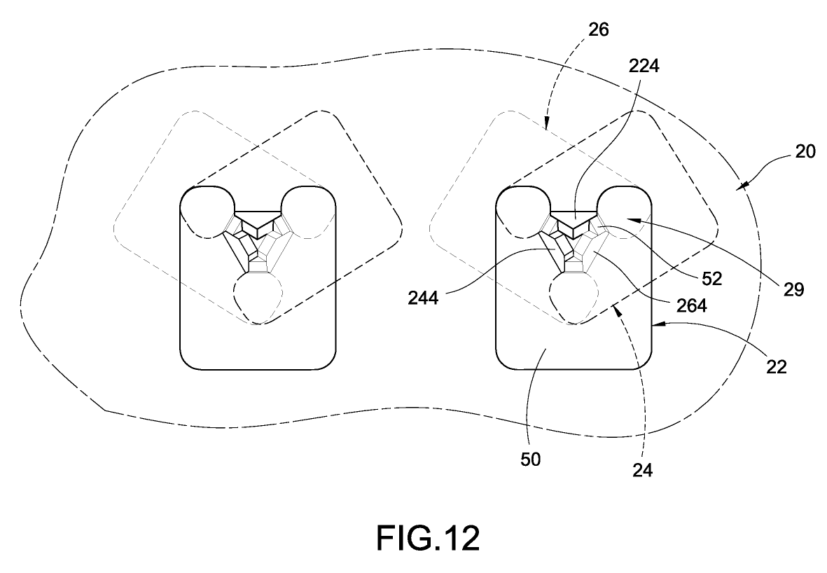

FIG. 12 shows a top view of an assembled view of a first sheet, second sheet and third sheet of the transdermal microneedle unit and a gasket according to an embodiment of the present invention. According to FIG. 12, the relationship of position of the first sheet 22, the second sheet 24 and the third sheet 26 as well as the gasket 50 is clear. The transdermal microneedle unit 20 has an opening 29 on the bottom through where the projecting part 52 of the gasket 50 may penetrate to seal the opening 29 effectively in order to avoid a leakage of medications.

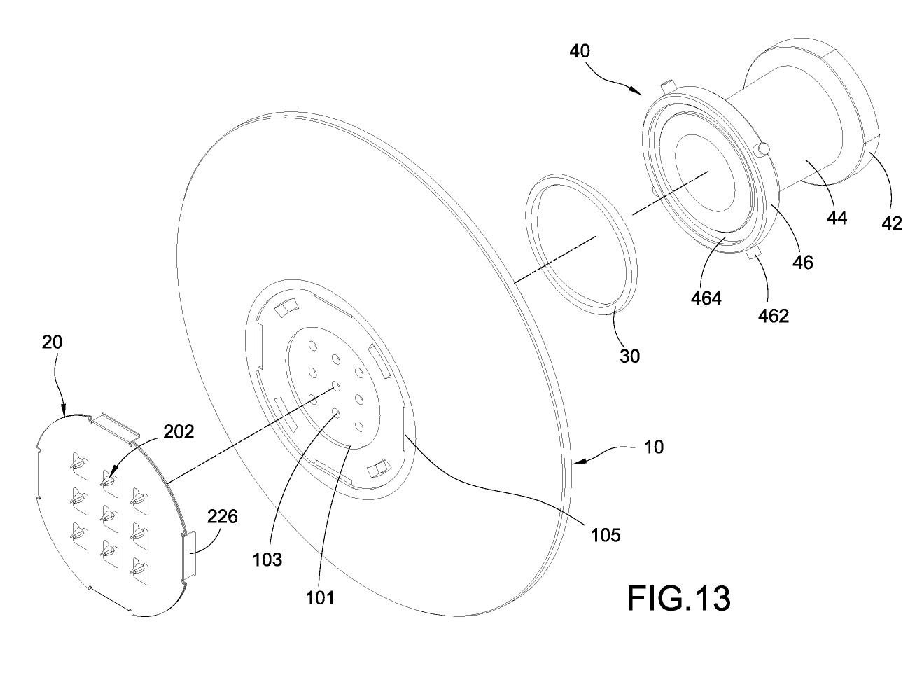

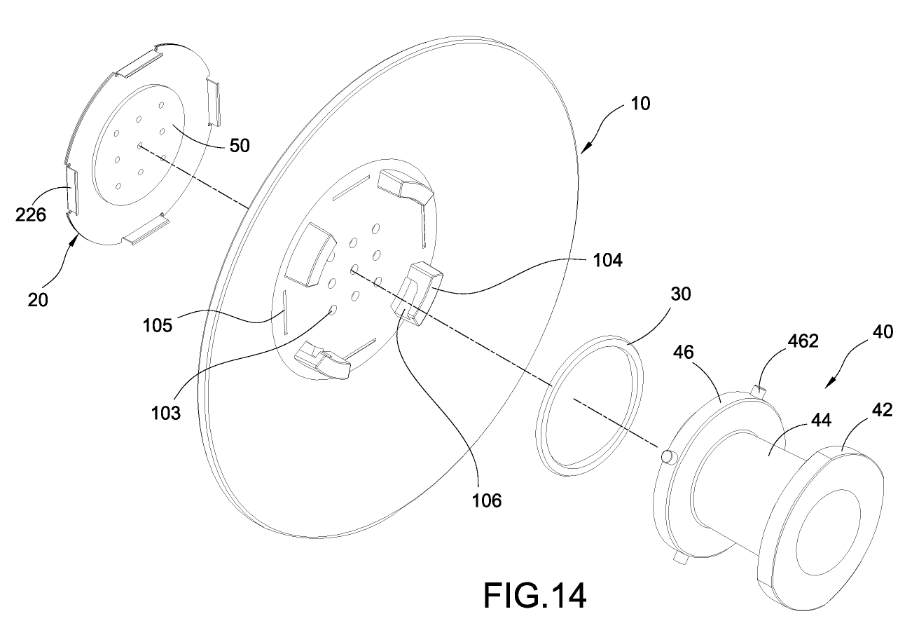

FIG. 13 shows an exploded view of a transdermal microneedle drug delivery device according to an embodiment of the present invention from a front viewing direction. FIG. 14 shows an exploded view of a transdermal microneedle drug delivery device according to an embodiment of the present invention from a rear viewing direction. In an embodiment, the transdermal microneedle drug delivery device comprises a substrate 10, a transdermal microneedle unit 20, an O-ring 30 and a union joint 40, wherein the O-ring 30 is provided on a front surface of a front end 46 of the union joint 40.

The substrate 10 has a plurality of holes 103 in a central region 101, in which the holes may be arranged in a 3×3 array to correspond to transdermal microneedles 202 with 3×3 array-arranged micro-needles of the transdermal microneedle unit 20. The substrate 10 has four grooves 105 at perimeter of the central region 101 for providing the transdermal microneedle unit 20. For example, the barbs 226 may be provided on the edge of the first sheet 22 to engage with the grooves 105 of the latter-mentioned substrate 10. In addition, the substrate 10 has a plurality of latches 104, and each of latches 104 has an entrance 106 at an end thereof. The union joint 40 has a plurality of projections 462 at a side surface of a front end 46. The union joint 40 may engage with the substrate 10 by screwing each of projections 462 into the corresponding entrances 106 of the latches 104.

As shown in FIG. 13, the union joint 40 has a front end 46, a middle section 44 and a rear end 42. The front end 46 of the union joint 40 has a circular groove 464 in the front surface for providing the O-ring 30 therein to avoid a leakage of medications. The union joint 40 may engage with the substrate 10 by the front end 46, and may engage with an injection syringe (not shown in FIGS. 13 and 14) by the rear end 42 for applying the medications into skin.

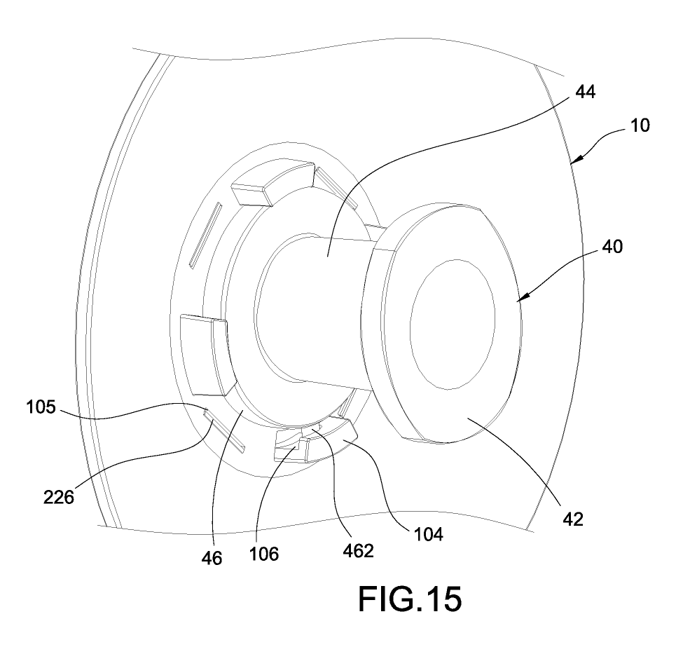

FIG. 15 shows an assembled view of a substrate and a union joint according to an embodiment of the present invention from a rear viewing direction. As shown in FIG. 15, the union joint 40 has a front end 46, a middle section 44 and a rear end 42. In addition, the substrate 10 has four latches 104, and each of latches 104 has an entrance 106 at an end thereof. The union joint 40 has four projections 462 at a side surface of a front end 46. The union joint 40 may engage with the substrate 10 by screwing each of projections 462 into the corresponding entrances 106 of the latches 104.

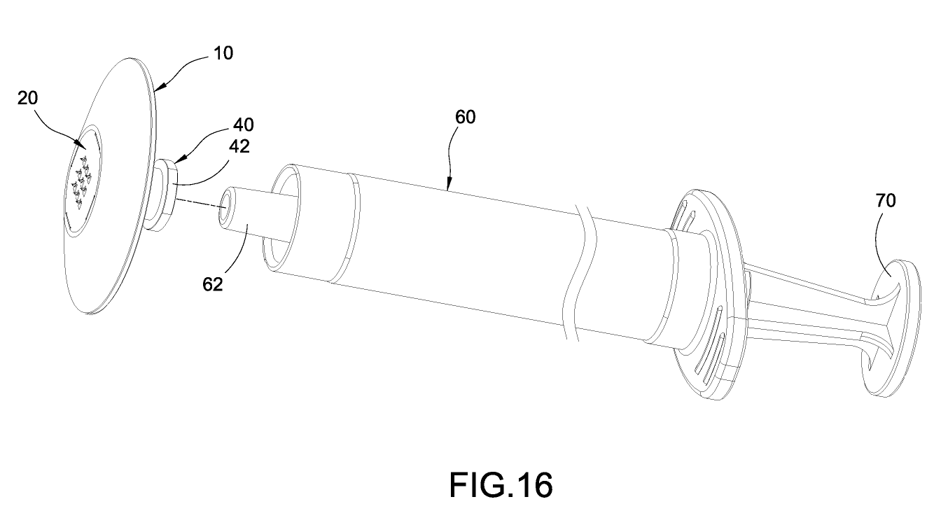

FIG. 16 shows a schematic view of a transdermal microneedle drug delivery device and an injection syringe in disassembled state according to an embodiment of the present invention. In an embodiment, the transdermal microneedle drug delivery device further comprises an injection syringe 60 including a plunger 70. The injection syringe 60 has a connecting end 62 for connecting with the rear end 42 of the union joint 40, and the plunger 70 can be pushed along inside a cylindrical tube of the injection syringe 60 to apply the medications into skin through the transdermal microneedle unit 20 engaged with the substrate 10. In operation, firstly a standard needle is connected to the injection syringe 60, and medication is drawn out from a medicine bottle by pulling the plunger 70 along inside a cylindrical tube of the injection syringe 60. Next, the standard needle is removed, and the transdermal microneedle unit 20 engaged with the substrate 10 is provided to apply the medication into skin. In an embodiment, the transdermal microneedle drug delivery device comprising an injection syringe 60 and a plunger 70 further includes prefilled medication so that it may directly apply the medication into skin.

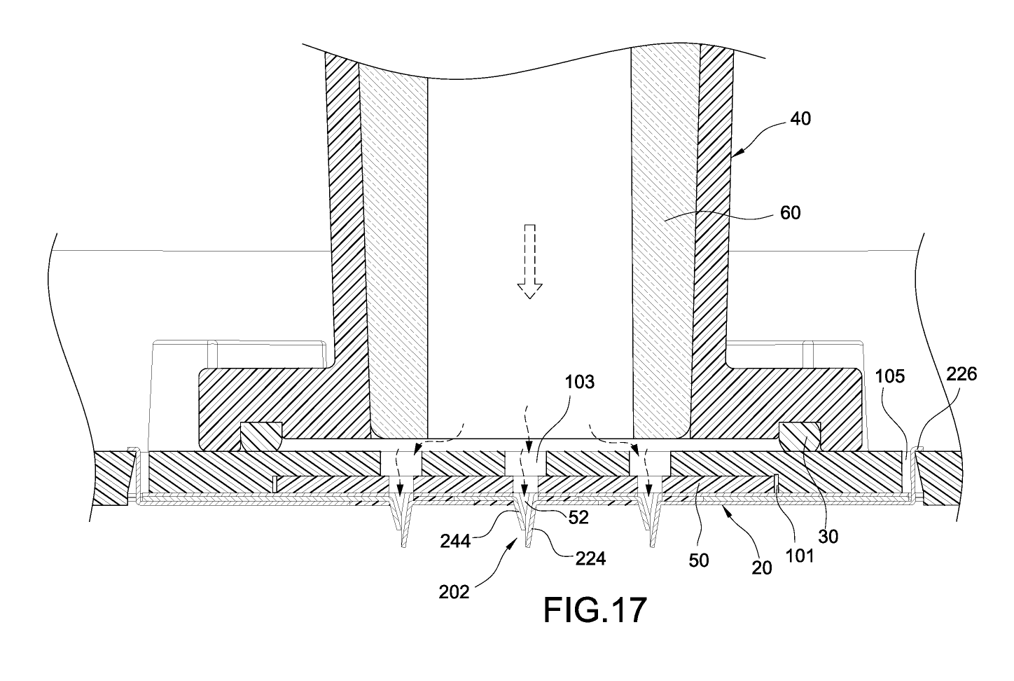

FIG. 17 shows a sectional assembled view of a transdermal microneedle drug delivery device and an injection syringe for applying drug according to an embodiment of the present invention. In an embodiment, the transdermal microneedle drug delivery device comprises a substrate 10, a transdermal microneedle unit 20 and a union joint 40. The substrate 10 has a plurality of holes 103 in a central region 101, in which the holes 103 may be arranged in an array to correspond to transdermal microneedles 202 with array-arranged micro-needles of the transdermal microneedle unit 20, wherein the transdermal microneedle 202 comprises a first barbule 224, a second barbule 244 and a third barbule (not shown in FIG. 17). The projecting part 52 of the gasket 50 may penetrate to seal the opening 29 on the bottom of the transdermal microneedle unit 20 effectively in order to avoid a leakage of medications. Also, the O-ring 30 is provided on a front surface of a front end of the union joint 40. The barbs 226 may be provided on the edge of the first sheet 22 to engage with the grooves 105 of the substrate 10. The injection syringe 60 has a connecting end 62 for connecting with the rear end of the union joint 40, and the plunger 70 can be pushed along inside a cylindrical tube of the injection syringe 60 to apply the medications into skin through the transdermal microneedle unit 20 engaged with the substrate 10.

In an embodiment, the transdermal microneedle drug delivery device may be used in a continue type or a close loop in accordance with mechanisms of drug metabolism of a patient. The accurate drug delivery of a close loop can be achieved in combination of a micro sensor of detecting the concentration of drug in the body of the patient.

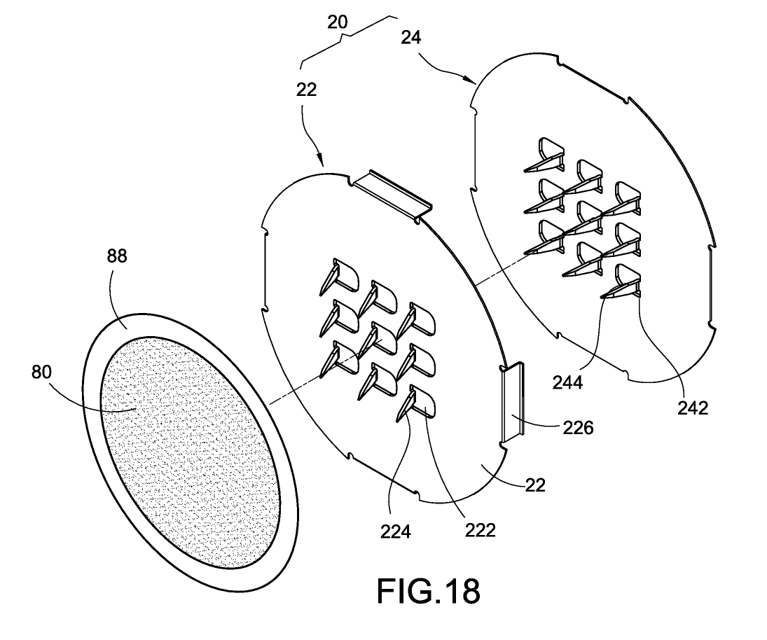

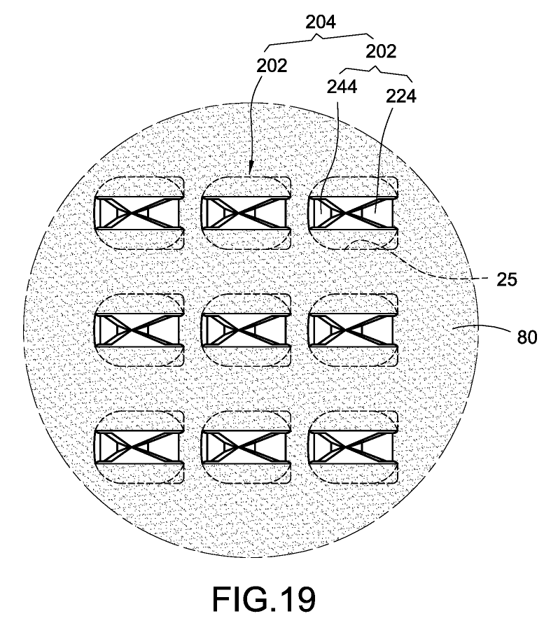

Please refer to FIGS. 18 to 20. The transdermal microneedle unit 20 of this embodiment is formed by stacking a first sheet 22 with a second sheet 24, and the first sheet 22 has a plurality of first through holes 222 formed thereon, and each first through hole 222 has a first barbule 224 disposed at the periphery of the first through hole 222, and a plurality of barbs 226 disposed at the periphery of first sheet 22, and the second sheet 24 has a plurality of array-arranged second through holes 242 formed thereon, and a second barbule 244 disposed at the periphery of each second through hole 242, wherein each first through hole 222 and each second through hole 242 arranged into an array, and the length L of the first barbule 224 and the second barbule 244 falls within a range of 0.6 to 1.5 mm and preferably within a range of 0.8 to 1.2 mm. During assembling, the first sheet 22 is stacked onto the corresponding second sheet 24, so that the second barbules 244 of the second sheet 24 pass through the first through holes 222 of the first sheet 22 respectively, and the second through holes 242 are communicated with the first through holes 222 respectively, and the first barbule 224 and second barbule 244 dispsoed in the same first through hole 222 constitute a transdermal microneedle 202 and an orifice 25 is formed at the outer boundary of the transdermal microneedle 202.

In this embodiment, the transdermal microneedle unit 20 further comprises an adhesive film 80, and a release paper 88 is attached onto a surface of the adhesive film 80, and the release paper 88 and adhesive film 80 are adhered to a surface of the first sheet 22 and cover each orifice 25, and each transdermal microneedle 202 is penetrated through the adhesive film 80 and release paper 88 to the outside.

During use, the release paper 88 is torn away from the adhesive film 80, and then the adhesive film 80 is adhered to a surface of human skin surface.

Further, the transdermal microneedle 202 is a semi-hollow cone; and each transdermal microneedle 202 is arranged separately to form an array-arranged transdermal microneedle 204.

Since cuticles or subcutaneous nerves of the skin of an infant or young child are closer to the exterior of the skin, therefore a thicker adhesive film 80 may be used in order to allow the tip of each transdermal microneedle 202 to be exposed and protruded from the adhesive film 80 by a length of 0.4 to 0.9 mm.

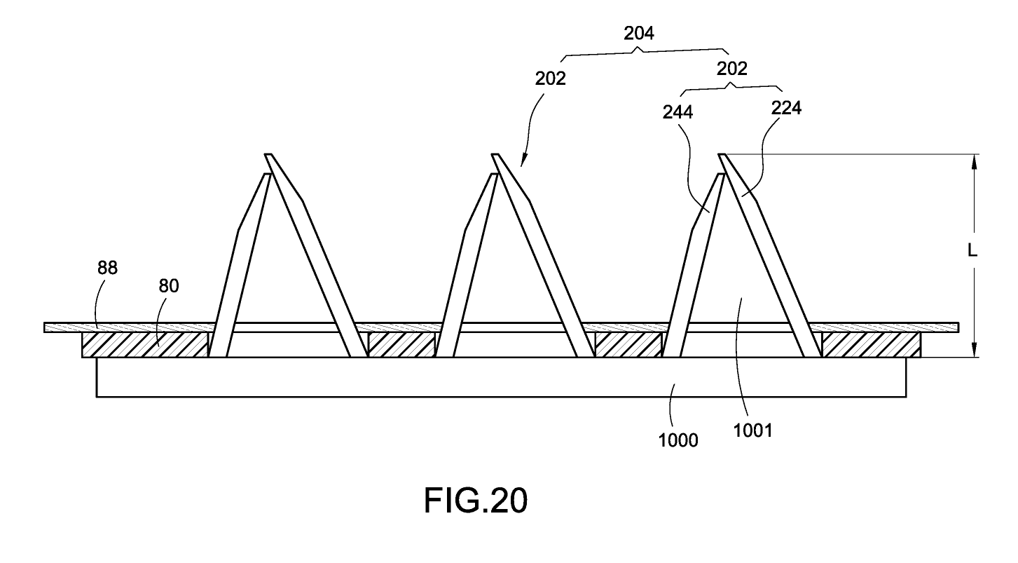

In FIG. 20, the transdermal microneedle unit 20 of this embodiment is combined with the substrate 1000, and there is a space 1001 surrounded by the barbules of the transdermal microneedle unit 20 for embedding with a dissolvable paste medication or dissolvable dry medication. The substrate 1000 can be an adhesive backing or a plastic plate.

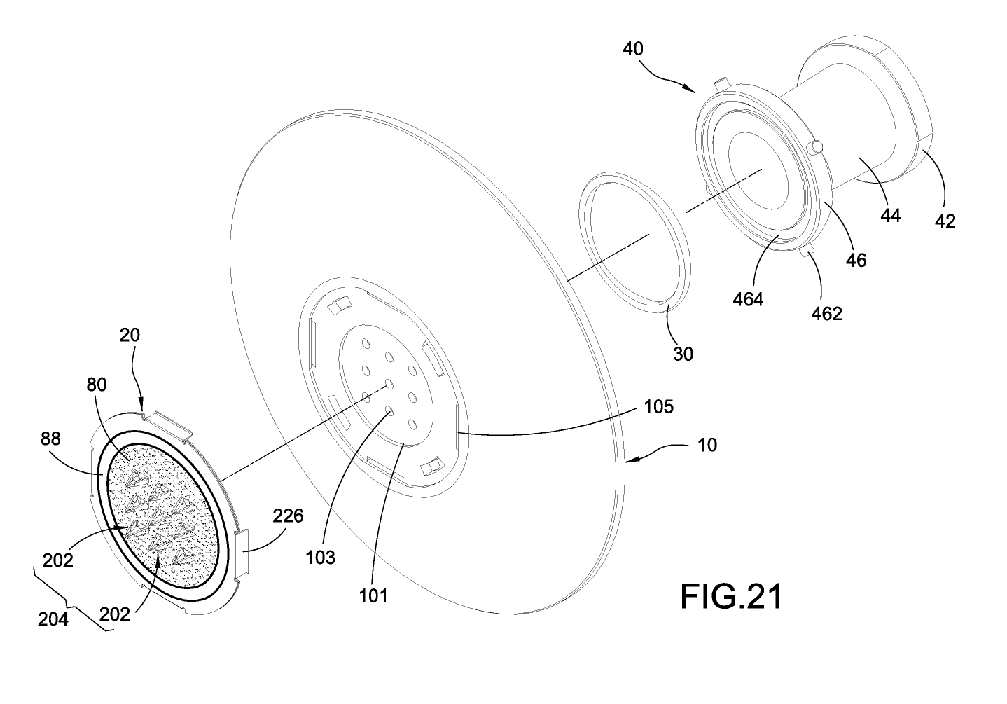

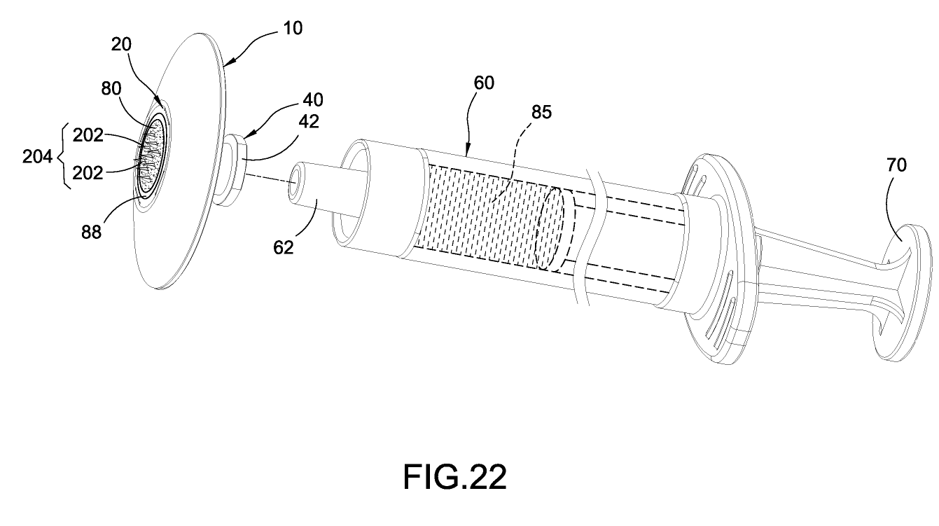

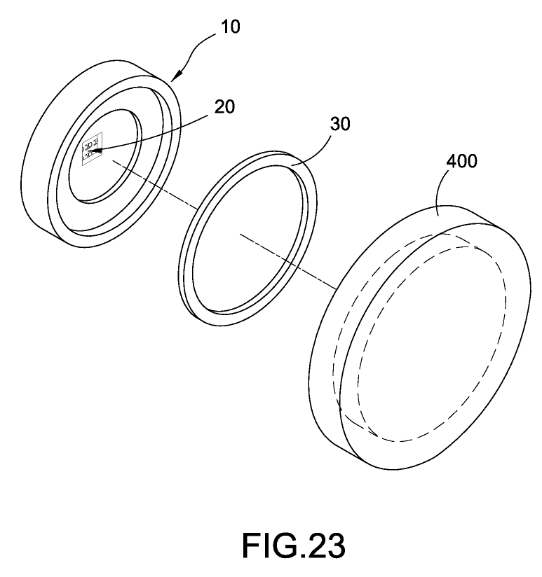

In FIG. 21, the transdermal microneedle unit 20 of this embodiment is combined with the substrate 10, O-ring 30 and union joint 40 to form a transdermal microneedle drug delivery device. The detailed structure and connection of the substrate 10, O-ring 30 and union joint 40 have been describe above, and thus will not be repeated. Please refer to FIG. 22 for a transdermal microneedle drug delivery device according to an embodiment of the present invention. The transdermal microneedle drug delivery device further comprises an injection syringe 60, a plunger 70 and a dissolvable paste medication 85, and the injection syringe 60 has a connecting end 62 coupled to a rear end 42 of the union joint 40, and the plunger 70 is plugged into the injection syringe 60 and provided for applying the paste medication 85 contained in the injection syringe 60 into a user's skin by a needle comprising the transdermal microneedle unit 20 and substrate 10.

Further, the paste medication 85 does not flow at room temperature, and has a coefficient of viscosity falling within a range from 10000 cP to 10000000 cP; or 10 Pa·s to 10000 Pa·s. Wherein, 1 Pa·s=1000 cP.

Currently, the only available FDA approved intradermal influenza vaccine is the BD Soluvia system. However, this system is associated with the same disadvantages as an intramuscular flu shot and mainly the requirement for cold chain. In contrast, a dry formulation in microneedle patches could offer practical benefits such as thermostability and independence of cold chain which is required for liquid formulations. However, the dissolving microneedles need to have enough mechanical strength to penetrate the skin, the ratio of the dissolvable excipient to drug in the microneedle should be higher to guarantee the microneedles have structural strength. In general, structural stability of dissolvable excipient formulations affects material form, structural strength, failure mode, diffusion and dissolution rate of dissolving microneedles.

On the contrary, in this invention, the present microneedle composed of at least two metal barbules can penetrate the skin easily, which allow drug between two barbules to have lower ratio of dissolvable excipient that can help the drug dissolve within the skin in minutes. Also, dissolvable drug/excipient can be used for delivery of nanoparticles, enabling a sustained-release of active agents to the diseased tissue.

In general, inherent structural stability of present invention can permit drug/dissolvable excipient formulations only focus on diffusion and dissolution rate. In one embodiment of the present invention, the drug between two barbules can be dry formulation or paste formulation of dissolving microneedle patch. The material of the dissolvable excipient can include sugar, polyacrylic acid (PAA), polyethylene glycol (PEG), polyethylene oxide (PEO), polyvinylpyrrolidone (PVP), polyvinyl alcohol (PVA), gamma-polyglutamic acid (γ-PGA), gelatin, maltose, xanthan gum and various water-soluble carbohydrate and derivatives thereof.

Preferably, the material of the dissolvable excipient for transdermal drug delivery according to the present invention can include chitosan, chitin, silk, carboxymethyl cellulose (CMC), chondroitin, collagen, gelatin, the foregoing crosslinked material, the foregoing derivatives, or polysaccharide derivative.

Preferably, the drug encapsulated in the micro-needle patch for transdermal drug delivery according to the present invention can include hydrophilic drugs or macromolecular drugs having a molecular weight greater than 500 Da, such as DNA (deoxyribonucleic acid), protein, vaccine, peptide, bacteria or chemical synthetic drug.

| | 4 × 4 microneedle patch | 5 × 5 microneedle patch |
|---|---|---|
| barbules | At least encapsulating | At least encapsulating |
| 0.8 mm height | 96 ug (excipient 46 ug + | 150 ug (excipient 100 ug + |
| 250 μm in width | drug* 50 ug) | drug* 50 ug) |
| at the base | | |
| barbules | At least encapsulating | At least encapsulating |
| 1.0 mm height | 96 ug (excipient 46 ug + | 150 ug (excipient 100 ug + |
| 300 μm in width | drug* 50 ug) | drug* 50 ug) |
| at the base | | |

Note
*: a single dose of inactivated influenza vaccine (fluvirin: 18 μg of haemagglutinin per H1N1 vaccine strain, 17 μg of haemagglutinin per H3N2 vaccine strain, and 15 μg of haemagglutinin per B vaccine strain)

In FIG. 23, the union joint 40 as shown in FIG. 22 may be replaced by an elastic cover 400, so that the transdermal microneedle unit 20 of this embodiment may be combined with the substrate 10, O-ring 30 (not shown in the figure) and elastic cover 400 to form a transdermal microneedle drug delivery device, and an end of elastic cover 400 is coupled to the substrate 10, and the other end of the elastic cover 400 is sealed. The detailed structure and connection of the substrate 10, O-ring 30 and elastic cover 400 have been described above, and thus will not be repeated.

During use, the paste medication 85 is put into the elastic cover 400, and the elastic cover 400 is pressed by a user's finger in order to apply the paste medication 85 into a user's skin without requiring the injection syringe 60 of FIG. 22. It is noteworthy that the elastic cover 400 may also be replaced by the conventional ontiment tube (whose operation is similar to squeezing toothpaste). Basically, the dose of the paste medication 85 per squeeze is determined by the space formed and enclosed by the array-arranged transdermal microneedles 204 and the cells surrounding the microneedle in this embodiment. In other words, the dose is determined by the quantity and size of the microneedles times the ratio of the main composition of the medication to the excipient.

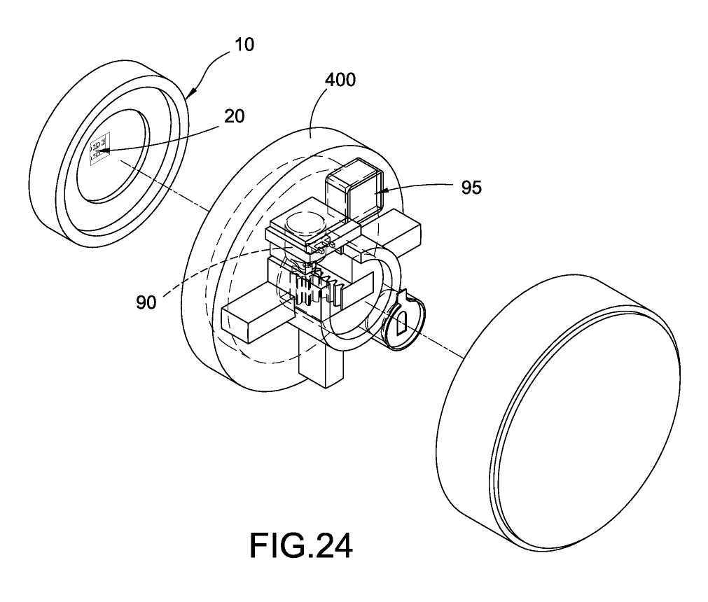

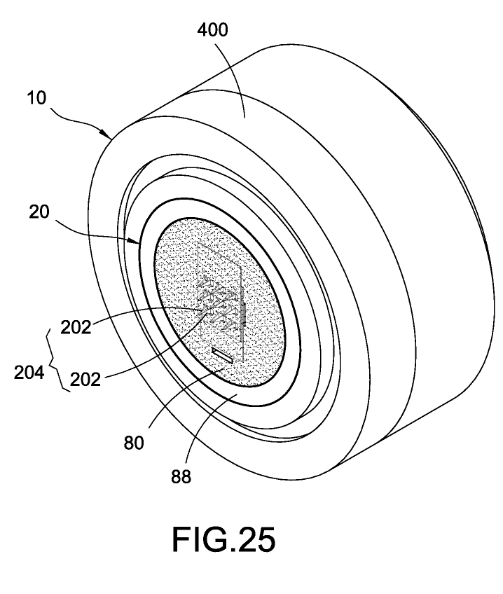

Please refer to FIGS. 24 and 25 for transdermal microneedle drug delivery device according to another embodiment of the present invention. The transdermal microneedle drug delivery device may further comprises a driving module 90 and a control module 95 added to the elastic cover 400 of FIG. 23, and the driving module 90 comprises a step motor, a gear mechanism, and a micropump, and the control module 95 comprises a micro control unit, and a battery. Wherein, the micropump, micro control unit and battery are coupled to the elastic cover 400 by the union joint 40, and a signal may be generated by the micro control unit and battery to drive the operation of the micropump in order to apply a paste medication 85 into a user's skin. This embodiment no longer needs to press the elastic cover 100 by the user's finger, and achieves the effect of automatically squeezing and delivering the medication.

The invention is not limited to these embodiments, but various variations and modifications may be made without departing from the scope of the invention.

What is claimed is:

1. A transdermal microneedle unit, comprising: at least two sheets stacked with each other, each of the sheets having a through hole defined thereon and a barbule arranged at a periphery of the through hole and having a length of 0.6 to 1.5 mm, wherein each of the through holes on one of the at least two sheets is penetrated by the respective barbules of another one of the at least two sheets, and any two of the barbules disposed in the same through hole constitute a transdermal microneedle and an orifice is formed at the outer boundary of the transdermal microneedle; the transdermal microneedle unit further comprises an adhesive film, adhered to one of the sheets and covering each of the through holes, and each of the transdermal microneedles penetrates through the adhesive film to the outside.

2. The transdermal microneedle unit in claim 1, wherein each barbule of the transdermal microneedle comprises a tip, and the tips of those barbules, after the sheets are stacked together, are not at the same altitudes to form an opening.

3. The transdermal microneedle unit in claim 1, wherein each barbule of the transdermal microneedle comprises a tip, and the tips of those barbules, after the sheets are stacked together, are at the same altitudes to form an opening by cutting at least one tip of the barbule.

4. The transdermal microneedle unit in claim 1, wherein the barbules of the transdermal microneedle has a length of 0.8 to 1.2 mm.

5. The transdermal microneedle unit in claim 1, wherein the transdermal microneedle is a semi-hollow cone, and each of the transdermal microneedles are arranged separately to form an array-arranged transdermal microneedle.

6. The transdermal microneedle unit in claim 1, wherein the transdermal microneedle unit is combined with a substrate, and there is a space surrounded by the barbules of the transdermal microneedle unit for embedding with a dissolvable paste medication or dissolvable dry medication.

7. A transdermal microneedle drug delivery device, comprising:
a substrate;
a transdermal microneedle unit, provided on the substrate, the transdermal microneedle unit comprising at least two sheets, each having a plurality of through holes, and each through hole having a barbule dispsoed at a periphery of the through hole and the barbule having a length falling within a range of 0.6 to 1.5 mm, and the through holes of one of the sheets being provided for passing the barbules of another sheet respectively, and any two of the barbules disposed in the same through hole constituting a transdermal microneedle and an orifice being formed at the outer boundary of the transdermal microneedle; and the transdermal microneedle unit further comprising an adhesive film, and the adhesive film being adhered to one of the sheets and covering each of the through holes, and each of the transdermal microneedles passing through the adhesive film to the outside periphery; and a union joint, connected with the substrate by an end thereof.

8. The transdermal microneedle drug delivery device in claim 7, wherein the substrate has a plurality of latches, and each of latches has an entrance at an end thereof, and the union joint has a plurality of projections at a side surface of an end, and the union joint is engaged with the substrate by latching the projections into the entrances of the latches respectively.

9. The transdermal microneedle drug delivery device in claim 8, wherein the union joint has a circular groove disposed on a distal end of the union joint.

10. The transdermal microneedle drug delivery device in claim 9, further comprising an O-ring installed in the circular groove of the union joint.

11. The transdermal microneedle drug delivery device in claim 7, further comprising an injection syringe and a plunger, and the injection syringe having a connecting end coupled to the other end of the union joint, and the plunger being plugged into the injection syringe to inject a dissolvable paste medication contained in the injection syringe into skin.

12. The transdermal microneedle drug delivery device in claim 7, further comprising a driving module and a control module, and the driving module being coupled to the other end of the union joint, and the control module generating and transmitting a signal to the driving module to inject a dissolvable paste medication into skin.

13. The transdermal microneedle drug delivery device in claim 12, wherein the dissolvable paste medication has a coefficient of viscosity falling within a range from 10000 cP to 10000000 cP.

14. The transdermal microneedle drug delivery device in claim 7, wherein the barbule has a length falling within a range of 0.8 to 1.2 mm.

15. The transdermal microneedle drug delivery device in claim 7, wherein the transdermal microneedle is a semi-hollow cone, and each of the transdermal microneedles is arranged separately to form an array-arranged transdermal microneedle.

16. A transdermal microneedle drug delivery device, comprising:
a substrate;
a transdermal microneedle unit, installed on the substrate, and comprising at least two sheets stacked with each other, and each of the sheets having a plurality of through holes, a barbule disposed at the periphery of each through hole, and having a length of 0.6 to 1.5 mm, wherein each of the through holes on one of the sheets is penetrated by the respective barbules of other sheets, and any two of the barbules disposed in the same through hole constitute a transdermal microneedle and an orifice is formed at the outer boundary of the transdermal microneedle; the transdermal microneedle unit further comprises an adhesive film, adhered to one of the sheets and covering each of the through holes, and each of the transdermal microneedles is penetrated through the adhesive film to the outside; and
an elastic cover, with an end coupled to the substrate, and the other end sealed, and the elastic cover having a dissolvable paste medication disposed therein, and an operation means being used for applying the paste medication into skin.

17. The transdermal microneedle drug delivery device in claim 16, wherein the operation means is to press the elastic cover by a user's finger.

18. The transdermal microneedle drug delivery device in claim 16, further comprising a driving module and a control module installed into a closed end of the elastic cover, and the transdermal microneedle drug delivery device is operated by the driving module and the control module to deliver the dissolvable paste medication automatically.
